(19) United States
(12) Patent Application Publication
(10) Pub. No.: US 2013/0197562 A1
(43) Pub. Date: Aug. 1, 2013

(54) Gastric Stretch Devices, and Methods for Treatment of Obesity

(71) Applicant: Medtronic, Inc., Minneapolis, MN (US)

(72) Inventors: John Burnes, Coon Rapids, MN (US); Warren Starkebaum, Plymouth, MN (US)

(73) Assignee: Medtronic, Inc., Minneapolis, MN (US)

(21) Appl. No.: 13/749,391

(22) Filed: Jan. 24, 2013

Related U.S. Application Data
(60) Provisional application No. 61/591,316, filed on Jan. 27, 2012.

(51) Int. Cl. A61F 5/00

(52) U.S. Cl. CPC A61F 5/003
USPC 606/191

(57) ABSTRACT

Medical devices, systems and methods are provided and are designed to stretch the stomach wall of a patient to treat obesity. Stretch devices attached to the stomach wall may trigger stretch receptors to expand the stomach into a state of apparent stretching, causing early onset of satiety thereby causing the patient to consume less food. Stretching of the stomach can be achieved by the attachment of stretch devices to the wall of the stomach. The devices may be expandable and contractible and, in some embodiments, may take the form of a device that expands following attachment to a wall of the stomach.

### CROSS-REFERENCE TO RELATED APPLICATIONS

This application is related to U.S. Patent Application 61/591,316, entitled “Gastric Stretch Devices, Systems and Methods for Treatment of Obesity” filed on Jan. 27, 2012, incorporated by reference herein in its entirety.

### BACKGROUND

The present disclosure relates to devices, systems and methods for treatment of obesity.

Obesity is a major health concern in the United States and other countries. A significant portion of the population is overweight with the number increasing every year. Obesity is one of the leading causes of preventable death. Obesity is associated with several co-morbidities that affect almost every body system. Some of these co-morbidities include: hypertension, hyperglycemia, heart disease, stroke, high cholesterol, diabetes, coronary disease, breathing disorders, sleep apnea, cancer, gallstones, and musculoskeletal problems. An obese patient is also at increased risk of developing Type II diabetes.

Multiple factors contribute to obesity, including physical inactivity and overeating. A variety of medical approaches have been devised for treatment of obesity. Existing therapies include diet, exercise, appetite suppressive drugs, metabolism enhancing drugs, surgical restriction of the gastric tract, and surgical modification of the gastric tract. In general, surgery is reserved for patients in whom conservative measures, such as monitoring caloric intake or controlling appetite with appetite suppressants, have failed. In addition, surgery is generally reserved for patients who are seriously, and sometimes morbidly, overweight.

There have been many surgical approaches to obesity. For example, some patients have received implantation of one or more bulking prostheses to reduce stomach volume. A bulking prosthesis resides within the stomach and limits the amount of food the stomach can hold, theoretically causing the patient to feel a sensation of satiety. U.S. Patent Application Publication No. 20030040804 to Stack et al., for example, describes a tubular prosthesis that is designed to induce sensations of satiety within a patient.

Another approach is restrictive surgery, which surgically makes the stomach smaller by removing or closing a section of the stomach. This procedure also reduces the amount of food the stomach can hold, causing the patient to feel full. U.S. Patent Application Publication No. 20020183768 to Deem et al., discloses various techniques for reducing the size of the stomach pouch to limit caloric intake, as well as to provide an earlier feeling of satiety.

Another surgical procedure to treat obesity is the gastric bypass procedure. In the gastric bypass procedure, the surgeon creates a small stomach pouch to restrict food intake and constructs a bypass of the duodenum and other segments of the small intestine. This procedure limits the amount of food that can be ingested and subsequently digested or absorbed.

Surgical procedures for treatment of obesity, such as those described above, tend to be highly invasive, and each form of surgery may involve complications. Restrictive surgery may entail a risk of vomiting, for example, and gastric bypass surgery may result in unpleasant consequences known as “dumping syndrome.”

U.S. Pat. No. 6,540,789 to Silverman describes a technique for treatment of obesity involving introduction of an implant material into the stomach wall in the vicinity of the pyloric sphincter to inhibit emptying of the stomach. Silverman also describes introduction of an implant material to reduce distensibility and contractility of the stomach.

U.S. Patent Application Publication No. 20100331617 to Forsell describes an operable stretching device implantable in an obese patient for stretching a patient's stomach wall via an external operation device.

U.S. Patent Application Publication No. 20050245957 to Starkebaum et. al. hereby incorporated by reference in its entirety, describes devices and methods designed to bias stretch receptors in the stomach wall of a patient to treat obesity. Biasing of the stretch receptors by pre-stretching induces early sensation of satiety, causing the patient to consume less food.

U.S. Patent Application Publication No. 20060173238 to Starkebaum, hereby incorporated by reference in its entirety, describes a dynamically controlled gastric occlusion device which may control the degree of gastric constriction of an occluding device based upon a monitored physiological parameter or based upon time. By dynamically controlling the degree of gastric constriction, the device limits the ingestion of food to reduce caloric intake so that the patient loses weight while permitting ingestion of caloric energy necessary to prevent malnourishment.

### SUMMARY

In general, the present disclosure relates to abdominal stretch devices and methods for treatment of obesity of a patient. Stretching an area of the gastrointestinal (GI) tract may induce an early sensation of satiety, causing a patient to consume less food. In accordance with the disclosure, stretching of the gastrointestinal tract can be achieved by attachment of stretch devices to the wall of the stomach. Stretch devices attached to the stomach wall may trigger stretch receptors to expand the stomach wall into a state of apparent stretching, causing early onset of satiety. In this manner, the devices are capable of discouraging excessive consumption of food without the use of appetite suppressant medications or more invasive surgical intervention. In various embodiments, the stretched condition of the stomach wall may activate stretch receptors to provide, in effect, an early warning system for cessation of meal consumption. Consequently, the devices according to the disclosure may counteract increased obesity and promote weight loss among obese patients.

Some aspects in accordance with principles of the present disclosure relate to a stretch device or a plurality of stretch devices which may be attached to a wall of the stomach to stretch the stomach wall.

In some embodiments, stretch devices may be attached to the inside or outside of the stomach wall at various locations (for example at the fundus or greater curvature) on or within any of the layers of the stomach for example, the serosal, muscle or mucosal layers of the stomach; or may be placed at any other location of the GI tract (for example the small intestine or pylorus).

In yet further embodiments, stretch devices may be adjustable and may be expandable and/or contractible and may comprise actuation means. Stretch devices may comprise self-actuation or mechanical actuation means. In some embodiments, stretch devices may comprise adjustable elements and may comprise attachment assemblies for attaching the adjustable elements to tissue. Adjustable elements and/or attachment assemblies may comprise materials which self-expand following attachment to the stomach. Adjustable elements and/or attachment assemblies may comprise materials, polymers or alloys comprising any of the following materials or material properties: smart, memory, shape memory, superelastic, pseudoelastic, self-expanding, and/or self-reverting. In this manner, stretch devices may be easily and quickly implanted without the need for more complex or complicated operating systems to control the degree of stretch of the stomach tissue to which the stretch device or devices are attached. Stretch devices and/or portions of stretch devices according to further embodiments may comprise biocompatible metals, alloys or polymers. Stretch devices and/or portions of stretch devices may also comprise biodegradable, bioabsorbable or bioresorbable materials. In some embodiments, stretch devices may comprise coatings which may enhance biocompatibility and/or aid in anchoring, attachment, placement or delivery of the devices.

Stretch device members or elements may take various forms or configurations and may comprise for example, adhesives, glues, stents, wires, filaments, coils, springs, telescoping elements, hooks, clips, sutures, anchors, pledgets, rivets, barbs, struts, screws, meshes, patches and/or sheets and may further comprise a unitary component or multiple components.

Stretch devices may be attached to the stomach wall at one or more locations and may be attached in various configurations. Stretch devices may be attached to the stomach wall at linear or non-linear spaced-apart locations and may be oriented in any direction along the stomach wall. For example, stretch devices may be configured to stretch the stomach wall along the contours of the stomach wall. Stretch devices may stretch tissue of the stomach wall in one or more directions and in more one or more dimensions (e.g. two-dimensionally or three-dimensionally) and may, for example stretch the stomach wall in a radial fashion.

Stretch devices in accordance with the present disclosure may be adapted for attachment to the stomach of a patient via placement devices and may be adapted to be delivered to locations along the stomach via laparoscopic or endoscopic surgical techniques. Systems according to some embodiments of the present disclosure may comprise laparoscopic or endoscopic delivery devices sized for introduction into a stomach of a patient via laparoscopic instruments or via the esophagus of a patient. A placement, tool and/or delivery device may hold a stretch device or devices or portions of a stretch device for delivery and attachment to the wall of the patient's stomach. As examples, the placement and/or delivery tools may take the form of a gripping member that grips a stretch device, or the placement tool may take the form of a device comprising a lumen such as a catheter, a hollow needle or a tube.

Other aspects in accordance with principles of the present disclosure relate to a method of stretching a portion of a gastrointestinal tract of a patient and may comprise attaching a stretch device or devices to a stomach wall of a patient to expand the stomach wall and thereby induce a sensation of satiety in the patient.

Various embodiments of the present disclosure are capable of solving at least some of the problems mentioned above. For example, devices, systems and methods in accordance with the present disclosure may provide a treatment for obesity that presents greater efficacy and lesser side effects relative to administration of conventional appetite suppressant medications. In some embodiments, devices of the present disclosure may be capable of being delivered laparoscopically or endoscopically and may thereby avoid the need for more highly invasive surgical procedures. Devices in accordance with the disclosure may thus be capable of avoiding substantial reconstruction of the stomach, and offer reduced damage, recovery time, and side effects.

The details of one or more embodiments of the present disclosure are set forth in the accompanying drawings and the description below. Other features, objects, and advantages of the disclosure will be apparent from the description and drawings, and from the claims.

### BRIEF DESCRIPTION OF THE DRAWINGS

FIG. 1 is a cross-sectional diagram of the interior of a stomach with stretch devices placed at various locations along the stomach wall.

FIG. 2A is a diagram of the exterior of a stomach with an embodiment of a stretch device and in a contracted condition attached to a stomach wall.

FIG. 2B is a diagram of the exterior of a stomach with an embodiment of a stretch device and in an expanded condition attached to a stomach wall.

FIG. 3A is a diagram of the exterior of a stomach with portion of another embodiment of a stretch device.

FIG. 3B is a diagram of another embodiment of a stretch device in a contracted condition.

FIG. 3C is a diagram the device of FIG. 3B in an expanded condition.

FIG. 4A is a diagram of the exterior of a stomach with portion of another embodiment of a stretch device attached to a wall of the stomach and in an expanded condition.

FIG. 4B is a diagram of another embodiment of a stretch device comprising a tether.

FIG. 4C is a diagram of the device of FIG. 4B with the tether engaged.

### DETAILED DESCRIPTION

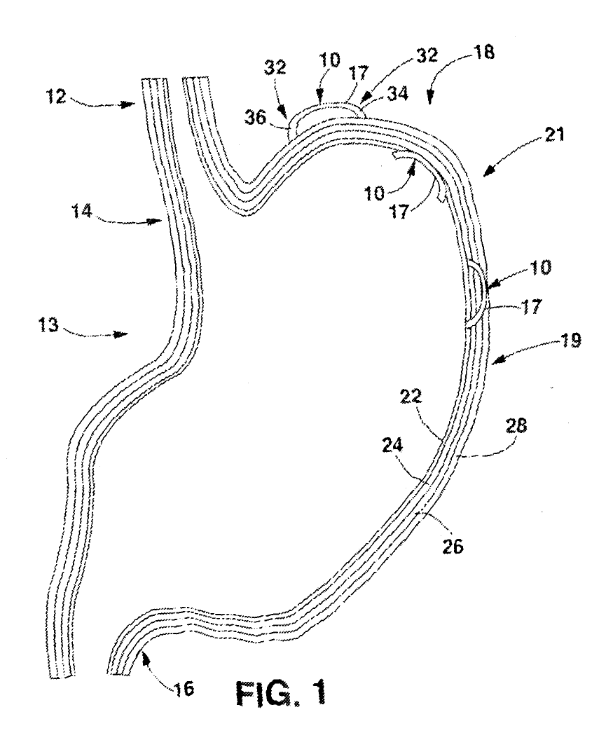

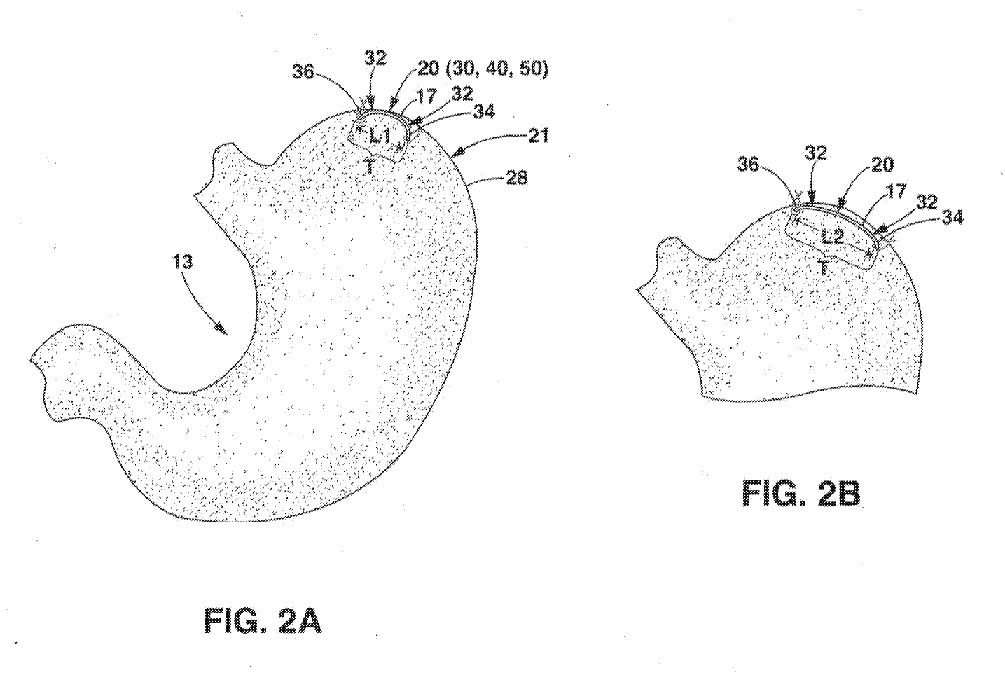

FIG. 1 is a cross-sectional diagram of the interior of a stomach 13, including esophagus 12, lower esophageal sphincter 14, pyloric sphincter 16, fundus 18, and greater curvature 19, with stretch devices 10 (three stretch devices 10 are depicted in FIG. 1) attached to stomach wall 21. Aspects of the present disclosure provide a stretch device 10 securable or implantable to a stomach wall 21. In general terms, stretch devices of the present disclosure, such as stretch devices 10—schematically illustrated in FIG. 1 as well as stretch devices 20, 30, 40 (FIGS. 2, 3, 4), may include an adjustable member 17 and one or more attachment assemblies 32 (referenced generally) which may be integrally formed with adjustable member 17 or may include component or components separate from adjustable member 17. It is to be understood that stretch devices 20, 30, 40 may comprise features described herein with specific reference to stretch device 10. As described in greater detail below, the adjustable member 17 is transitionable from a contracted state prior to implantation to an expanded state following implantation. The attachment assembly 32 can assume various forms, described below, that are capable of securing an adjustable member 17 to the stomach wall 21 in a manner such that upon transitioning of the adjustable member 17 to the expanded state following implantation, the stretch device 10 transitions to an expanded condition and causes stretching of stomach stretch receptors. While FIG. 1 illustrates three stretch devices 10, any other number is equally, acceptable, for example, only one stretch device 10, or more than one stretch devices 10.

Stretch devices 10 (also 20, 30, 40) as hereinafter described with reference to FIGS. 2, 3, and 4) can be implanted within or attached to the outside of the stomach 13, the inside of the stomach 13 and/or within portions of the stomach wall 21. Stomach wall 21 of a human stomach 13 generally includes four layers. With reference to FIG. 1, the innermost layer, mucosa 22, generates digestive juices. Submucosa 24 contains blood vessels that provide blood and oxygen to mucosa 22. Muscularis 26, a smooth muscle layer embedded with nervous plexus, contracts to mix food with digestive juices generated by mucosa 22. Serosa 28, the fourth and outermost layer, protects the other layers and confines digestive juices to stomach 13.

Stretch device 10 or portions of stretch device 10 may be attached to the serosa, the mucosa, the submucosa or the muscularis 26, which contains the stretch receptors. The stretch receptors are coupled to the nervous system via the vagus nerves, and signal the patient when stomach 13 reaches a stretch point indicating a large quantity of food. With stretch device 10 attached and expanded, the patient perceives that the stomach has reached a stretch point indicating fullness much earlier during the course of the meal and at a point at which the stomach is not actually full. Stretch device 10 may be attached to or may embed in only one or several or portions of the layers of the stomach wall 21. Stretch device 10, for example, may attach to the stomach wall 21 at the serosa 28 at at least two spaced apart locations. Stretching or expansion of stretch device 10 following attachment to the stomach tissue may cause the tissue or layers of the stomach tissue T between the areas of attachment of stretch device 10 to expand as adjustable member 17 and thereby device 10 expands and may thus expand stretch receptors in the stomach wall 21 and induce a sensation of satiety in a patient.

As further shown in FIG. 1, stretch device 10 may be attached to the stomach wall 21 at the fundus 18 at spaced apart positions to provide localized stretching at several different points. Localized stretching may occur in more than one dimension, for example, localized stretching may occur two-dimensionally or three-dimensionally (e.g., along the contours of stomach wall 21). Stretch device 10 may be attached to stomach wall 21 in other regions of stomach 13, other than fundus 18, such as greater curvature (e.g. stretch device 10C). However, stretch receptors tend to be concentrated within fundus 18. Accordingly, in some embodiments, stretch device 10 may be primarily or solely attached to fundus 18, where they are expected to be most effective in expanding or stretching stretch receptors. In other words according to some embodiments, stretch device 10 may be generally located only at the region of fundus 18 and nowhere else. In other embodiments, stretch device 10 may be attached to fundus 18 and greater curvature 19, or solely at the region of greater curvature 19.

Adjustable member 17 (referenced generally) may be formed from a smart memory, shape memory, superelastic, pseudoelastic, self-expanding, and/or self-reverting material, alloy or polymer. Following attachment of an adjustable member 17 to a location along the stomach 13, adjustable member 17 may assume an expanded or undeformed state thereby transitioning stretch device 10 to an expanded condition. The expanded state of adjustable member 17 may be described as a memory-set or shape memory set state. In some embodiments adjustable member 17 may comprise biocompatible materials of alloys which are not shape memory materials. With this construction, following attachment of stretch device 10 to tissue, stretch device 10 may be mechanically expandable upon attachment such as via a spring-like mechanism. As described above, attachment assembly 32 may be integrally formed with adjustable member 17 and may comprise the same material as adjustable member 17. Alternatively, attachment assembly 32 may comprise materials different from adjustable member 17. Attachment assembly 32 may comprise shape memory materials as described above and may transition from a first delivery state to a second attachment state separate from the transitioning of adjustable member 17 from a contracted state to an expanded state.

Stretch device 10 may be expanded to a condition with a size sufficient to expand stretch receptors within stomach wall 21. When expanded in this way, stretch device 10 exerts a localized stretching force on stretch receptors in stomach 13. The stretch receptors are coupled to the enteric nervous system of a patient. When triggered by the stretching force, the stretch receptors induce a sensation of satiety in the patient, and discourage the patient from consuming an excessively large meal. The role of stretch receptors in human gastric function is discussed, for example, in A. S. Paintal, “A study of gastric stretch receptors; their role in the peripheral mechanism of satiation of hunger and thirst,” J. Physiol. Nov. 29, 1954; 29; 126(2):255-70.

Stretch device 10 stretches stomach wall 21 into a stretched condition that either triggers the stretch receptors or causes earlier triggering of the stretch receptors during the consumption of a meal. Hence, even though the stomach may not contain a substantial portion of food at the outset of a meal, implanted or attached stretch device 10 has already stretched the stretch receptors into a condition that simulates the presence of a substantial portion of food. Consequently, during the course of a meal, stomach 13 requires a smaller amount of food to produce a sensation of satiety, which causes the patient to stop eating.

Stretch device 10 is configured such that even in an expanded state, the device does not significantly change the overall size or contents of stomach 13, but provides a localized modification of the stomach wall 21. This modification affects the response of the patient's enteric nervous system and the amount of food consumed by the patient, thereby preventing increased obesity and possibly causing or assisting in weight loss. In some cases, stretch device 10 may be explanted after a desired course of obesity treatment has been achieved.

Stretch device 10 may be implanted or attached surgically from the serosal aspect of stomach 13 (i.e., from the outer surface) or endoscopically from the mucosal aspect of the stomach (i.e., from the inside surface) of the stomach. The esophagus 12 of the patient may be intubated with the endoscopic delivery device via the oral or nasal passage under general anesthesia. Additionally, surgical implantation may involve laparoscopic techniques. In this manner, a highly invasive surgery can be avoided, and recovery time can be shortened.

One embodiment of a stretch device (stretch device 20) is shown in FIGS. 2A and 2B and generally includes an adjustable member 17 and at least one attachment assembly 32. FIG. 2A is a diagram of the exterior of stomach 13 with an adjustable member 17 of stretch device 20 in a first state and attached to the stomach wall 21. As shown in FIG. 2A, adjustable member 17 in a first state may comprise a deformed, contracted, constrained, delivery, and/or shortened state. With continued reference to FIG. 2A, adjustable member 17 may comprise a first end 34 and a second end 36. One or both ends 34, 36 may comprise an attachment assembly 32 for attaching adjustable member 17 to tissue. Adjustable members 17 may comprise, as several non-limiting examples: wire, filaments, coils, stents, springs, telescoping elements; patches, meshes, struts and/or sheets. Attachment assemblies 32 may comprise, as several non-limiting examples: adhesives, glues, wires, filaments, coils, springs, hooks, clips, sutures, anchors, pledgets, rivets, barbs, screws. As shown in FIG. 2A, opposing ends 34, 36 of adjustable member 17 may be implanted or attached at spaced apart positions along or at fundus 18 of stomach 13.

As depicted in FIG. 2A, adjustable member 17 may comprise a longitudinal length comprising a first, contracted length L1 and, following attachment to tissue of stomach 13, adjustable member 17 (and thereby device 20) may expand in a longitudinal direction to a second state which may comprise an expanded longitudinal length L2 as depicted in FIG. 2B. As further depicted in FIG. 2B, adjustable member 17 in a second state may comprise an undeformed, expanded, unconstrained, or lengthened state. Adjustable member 17 may be configured to self-expand to the second state. For example, adjustable members 17 and/or attachment assemblies 32 may comprise self-expanding springs or may comprise various shape memory materials or alloys and thus may self-assume (e.g. self-expand, self-revert) an undeformed, shape memory-set or memory-set configuration. Shape memory materials (also described, for example, as smart materials or memory materials) or alloys can exhibit pseudoelastic or superelastic behavior when deformed at a temperature slightly above a transformation temperature. The transformation temperature may be defined as the temperature at which a shape memory alloy finishes transforming from martensite to austenite upon heating (i.e., Aₜ temperature). At least a portion of the shape memory material or alloy is converted from its austenitic phase to its martensitic phase when the material or alloy is in its deformed configuration. As the stress or constraint is removed, the material undergoes a martensitic to austenitic conversion and springs back to its undeformed (e.g. unconstrained, expanded) configuration. In order for the pseudoelastic material to retain sufficient expansive force in its undeformed or expanded configuration, the material should not be stressed past its yield point in its deformed or contracted configuration to allow complete recovery of the material to its undeformed configuration. Shape memory materials or alloys may include additional elements which affect the yield strength of the material or the temperature at which particular pseudoelastic or shape transformation characteristics occur. Shape memory materials or alloys may also be heat activated or may comprise a combination of heat activation and pseudoelastic properties to allow the material to assume an undeformed state as is well known by those skilled in the art. One such shape memory alloy which adjustable members 17 (and/or attachment assemblies 32) may comprise is nitinol. Other shape or smart memory alloys or polymers as are known in the art may also be utilized.

FIGS. 2A and 2B depict an anterior side of stomach 13 for ease of illustration. It should be understood, however, that a stretch device (10, 20, 30, 40) or devices as depicted on the anterior side, may likewise be implanted on or attached to a posterior side of stomach 13. In other embodiments, stretch devices (10, 20, 30, 40) may be implanted on or attached to a single side or two sides, i.e., posterior, anterior, and/or lateral. In each case, stretch devices (10, 20, 30, 40) are implanted as relatively small contracted objects that then expand following implantation or attachment, and thereby stretch the stretch receptors in fundus 18 of stomach 13. Portions of a stretch device or devices (10, 20, 30, 40) may embed or attach to one or more layers of the stomach wall 21.

In the examples of FIGS. 1-4, adjustable members 17 are depicted as essentially crescent-shaped elements. However, in other embodiments, adjustable members 17 may comprise a variety of other shapes, e.g., substantially spherically shaped, rod or cylinder-shaped, sheet-like, curved surfaces, or irregularly shaped and may comprise a variety of different forms (e.g., wires, coils, springs, stents, sheets, patches, meshes and the like) as previously described above with reference to FIG. 2A. In some embodiments adjustable members 17 are configured for attachment to the stomach wall 21 such that when adjustable members 17 are in an undeformed, expanded, unconstrained, or lengthened state, the stomach wall 21 is stretched in more than one dimension. Adjustable members 17 may comprise a single member or a series of elements joined or attached together. In some embodiments, adjustable member 17 may comprise multiple elements arranged in a telescoping manner to allow for expansion and contraction of the adjustable member 17 and thereby cause expansion and contraction of device 10. Adjustable member 17 may be contracted or constrained in several ways for delivery and attachment of device 10 to a targeted area of tissue. In this manner, a system (not shown) may comprise a stretch device 10 and a placement tool (not shown) for delivery and attachment of stretch device 10 to the stomach. Placement tool (no shown) may take various forms and may for example comprise a holding member, a gripping member, a hollow tube or a catheter (not shown). Devices 10 and/or placement tool may be insertable into endoscopic or laparoscopic surgical tools for less invasive delivery of stretch device 10. Stretch device 10 may comprise a configuration which may conform to a stomach wall 21 and may comprise a low profile as compared to the tissue area to which the device 10 is attached.

The amount of stretch the stomach undergoes after attachment and expansion of stretch device 10 may be dependent upon the patient and may for example, be an amount of stretch sufficient to create a sensation of satiety sufficient to discourage the patient from consuming an excessively large meal. In some embodiments the stretch ratio of the stomach tissue from an at least partially stretched tissue state may be at least approximately two times and more particularly may be approximately twenty times. In some embodiments the amount of stretch of tissue of the stomach may comprise an amount sufficient to create a hormonal or neuronal response thereby inducing a sensation of satiety in the patient.

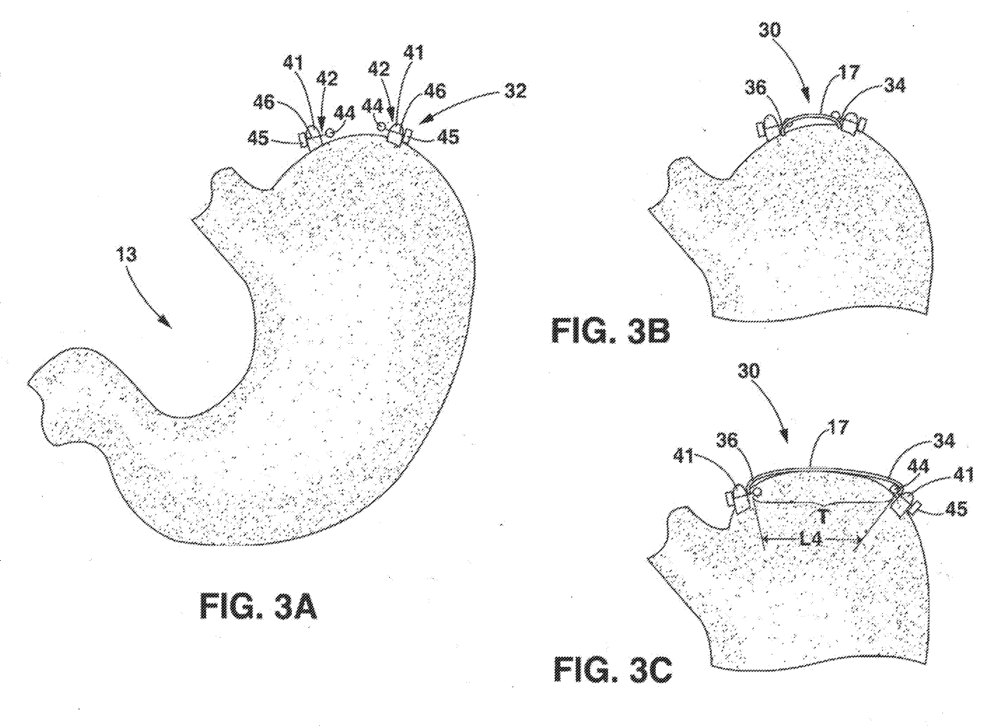

Another embodiment of a stretch device (stretch device 30) is shown in FIGS. 3A-3C and generally include an adjustable member 17 and at least one attachment assembly 32 comprising plication anchors 42. Adjustable members 17 may take any form as described above. The anchors 42 are described in greater detail below and are generally configured to promote attachment of the adjustable member 17 to the stomach wall 21. Stretch device 30 is shown on the outside of stomach 13. It is to be understood that device 30 may be attached to various locations along the stomach wall 21 as described herein with reference to stretch device 10 and may be attached to the inside of the stomach wall. With reference to FIG. 3A, a portion of stretch device 30 is depicted and shows an embodiment of attachment assemblies 32 comprising plication anchors 42 placed at two spaced apart locations on stomach 13. Tissue plications 41 are created by gathering or pinching stomach tissue together and anchoring the plication 41 with a plication anchor 42. Tissue plications 41 may be formed using a vacuum source (not shown) to suction tissue to be pinched together to create a plication 41. U.S. Pat. No. 6,592,596 to Geitz, incorporated by reference herein in its entirety, describes devices and methods for creating and securing a tissue fold, for example securing a region of the stomach circumferential to the esophageal opening back onto the esophagus, during an endoluminal medical procedure to alleviate the effects of Gastroesophageal Reflux Disease. Other methods of creating tissue plications may also be utilized, for example, mechanically pinching or holding tissue together with surgical tools, as is known in the art. Each of plication anchors 42 may comprise a plication anchor first end 44 and a plication anchor second end 45. The plication anchor first ends 44 may be configured to attach to an adjustable member 17 as depicted in FIG. 3B. Plication anchor 42 may comprise a cross member 46 which may pierce tissue of tissue plication 41 such that an anchor second end 45 abuts tissue plication 41 at first tissue plication end while an anchor first end 44 abuts tissue plication 41 at a second tissue plication end to thereby secure the tissue plication 41 between ends anchor ends 44 and 45. Plication anchor 42 may comprise a single component or multiple components. For example, plication anchor first and/or second ends 44, 45 may be releasably attached to plication anchor cross member 46. Alternatively plication anchor first and/or second ends 44, 45 may be integrally formed with cross member 46. Plication anchor 42 may be formed of any suitable material that is biocompatible. Plication anchor 42 may be formed of a shape memory material as described herein with reference to stretch devices 10. Plication anchor 42 or portions of plication anchor 42 may comprise a first state and a second state and may self-assume a second state upon attachment to tissue. Plication anchor first and second ends 44, 45 may comprise a variety of configurations and for example, may take the form of a circular, semi-circular rectangular, pointed, piercing, blunt or other object and may, for example, comprise a pledget. Plication anchor first and second ends 44, 45 may act in concert to compress gastrointestinal tissue therebetween. The compressive force of plication anchor 42 may result from the material properties (e.g. shape memory materials) or construction of plication anchor 42. Plication anchor 42 may be configured in a manner so as to allow plication anchor cross member 46 to be adjustable. For example, plication anchor 42 may comprise a spring or folded material and/or may be adjustable. Plication anchor 42 additionally may be expandable and/or contractible.

As depicted in FIG. 3A, two plication anchors 42 are shown with tissue plications 41 formed between plication anchor ends 44, 45. In FIG. 3B, an adjustable member 17 is depicted as attached to plication anchor end 44 at each of adjustable member ends 34, 36 while adjustable member 17 is in a first contracted state and has a length L3 in the first, contracted state. With reference to FIG. 3C, adjustable member 17 is depicted in an expanded or second state having an expanded length L4. With continued reference to FIG. 3C, the tissue, T of stomach wall 21, located between plications 41 is stretched as stretch device 30 is expanded to the length L4. The amount of stretch of tissue T may be as described previously herein and thus may be an amount sufficient to create a sensation of satiety in a patient. It is to be understood that any number of plication anchors 42 may be placed along the stomach wall to form any number of tissue plications 41. Likewise, any number of adjustable members 17 may be attached to or between plication anchors 42.

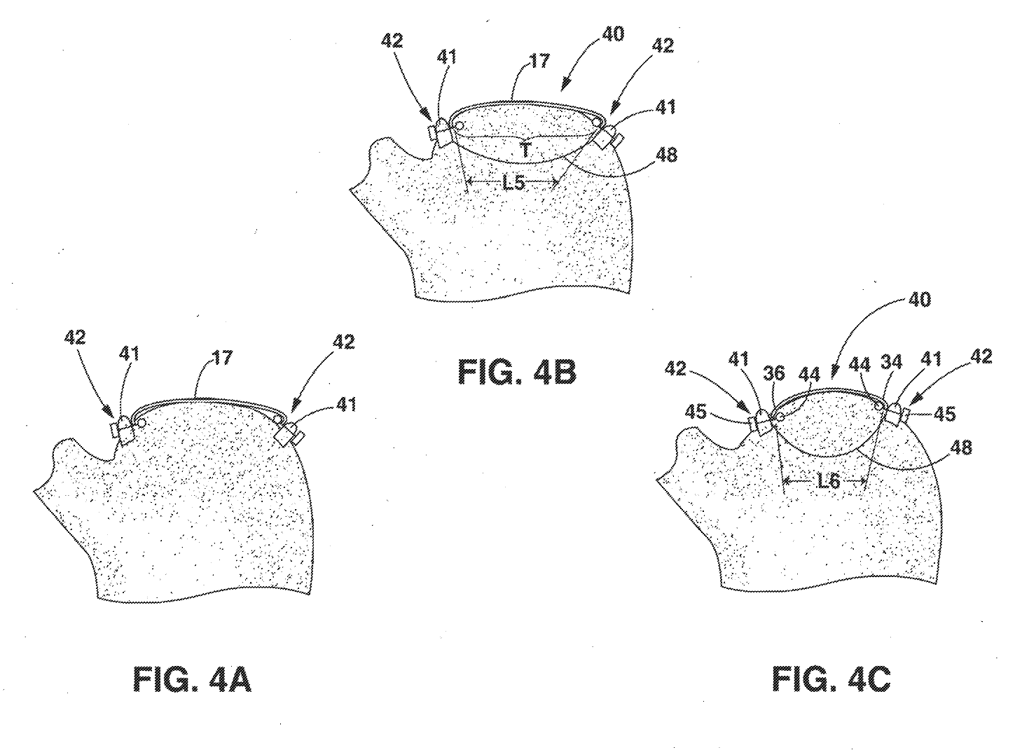

Another embodiment of a stretch device (stretch device 40) is shown in FIGS. 4A-4C and generally includes an adjustable member 17 and at least one attachment assembly 32 and further includes a tether 48, described in greater detail below. FIG. 4A depicts a portion of stretch device 40 and illustrates adjustable member 17 in a second expanded state and attached to a stomach wall 21 between plication anchors 42 as previously described herein. FIG. 4A depicts plication anchors 42 as the attachment assemblies 32. It is to be understood that device 40 may comprise any other of the attachment assemblies 32 as described above. In FIG. 4B, a tether 48 is attached to adjustable member 17. Tether 48 is generally configured to adjust or aid in adjusting adjustable member 17. Alternatively tether 48 may also be attached to plication anchors 42 at either of plication anchor first and second ends 44, 45 or to plication anchor cross member 46. Alternatively, tether 48 may be attached to tissue plication 41. Tether 48 may be expandable and/or contractible and may be constructed of any suitable biocompatible material and may comprise for example metals and/or polymers as described previously herein. Tether 48 may be tightened or foreshortened to reduce an adjustable member 17 expanded length L5 and thereby reduce an amount of stretch of tissue T along the stomach wall 21. Tether 48 may be actuated such as via a squiggle motor (not shown) to gather in or let out a length of tether 48. Gathering in of tether 48 may thereby reduce the length L5 of expanded adjustable member 17 to a contracted adjustable member length L6.

Stretch devices 10, 20, 30, 40 according to the disclosure may additionally comprise electrodes at various locations for stimulation of stomach tissue. U.S. Patent Application Publication No. 20110071589 to Starkebaum et. al, incorporated by reference herein in its entirety, describes systems, devices and techniques for delivering electrical stimulation therapy to a patient.

Although the present disclosure has been described with reference to embodiments, workers skilled in the art will recognize that changes can be made in form and detail without departing from the spirit and scope of the present disclosure.

What is claimed is:

1. A device for treatment of obesity, the device comprising:
an adjustable member configured to attach to a wall of a stomach of a patient;
wherein the adjustable member is transitionable between a first, contracted state and a second expanded state;
wherein the adjustable member is configured to attach to the stomach wall in the first contracted state and is configured to expand to the second expanded state after attachment to the stomach wall; and
wherein the adjustable member in the second expanded state is configured to stretch the wall of the stomach.

2. The device according to claim 1 wherein the second expanded state further comprises a shape memory set state and wherein the adjustable member is configured to self-revert to the shape memory set state.

3. The device according to claim 1 wherein the adjustable member comprises a material selected from the group consisting of: superelastic, pseudoelastic, self-expanding, self-reverting, shape-memory, and memory-set.

4. The device according to claim 3 wherein the adjustable member comprises nitinol.

5. The device according to claim 1 wherein the device comprises one of: a stent, a wire, a filament, a coil, a spring, a hook, a clip, a suture, an anchor, a pledget, a rivet, a barb, a patch and a sheet.

6. The device according to claim 1 wherein the adjustable member comprises at least two components.

7. The device according to claim 6 wherein the adjustable member comprises a telescoping device.

8. The device according to claim 1 wherein the adjustable member comprises a first end and a second end and wherein each of the first and second end comprise an attachment assembly for attaching the adjustable member to the stomach wall.

9. The device according to claim 8 wherein each attachment assembly comprises one of: a hook, a clip, a staple, a clamp, a suture, an anchor, a pledget, a rivet and a barb.

10. The device according to claim 8 wherein each attachment assembly is configured to self-actuate to attach the adjustable member to the stomach wall.

11. The device according to claim 8 wherein the device further comprises a tether.

12. A system for treatment of obesity of a patient, the system comprising:
a stretch device comprising an adjustable member transitionable between a first, contracted state and a second expanded state;
a placement tool configured to hold the stretch device for attachment to a wall of a stomach of the patient, the placement tool configured to hold the stretch device in the first state.

13. The system according to claim 12 wherein the second, expanded state further comprises a shape memory-set configuration and further wherein upon release of the stretch device from the placement tool the adjustable member resumes the shape memory-set configuration.

14. A method for treatment of obesity, the method comprising:
attaching a device comprising an adjustable member in a contracted state to a wall of a stomach of a patient and allowing the adjustable member to self-revert to an expanded state to thereby stretch the stomach wall.

15. The method according to claim 14, further comprising laparoscopically attaching the device to the stomach wall.

16. The method according to claim 14, further comprising endoscopically attaching the device to the stomach wall.

17. The method according to claim 14, wherein the device is configured to stretch the stomach wall an extent sufficient to stretch receptors in the stomach wall and thereby induce a sensation of satiety in the patient.

18. The method according to claim 17, wherein the device comprises nitinol.

19. The method according to claim 18, wherein the device further comprises an attachment assembly for attaching the adjustable member to the stomach wall.

20. The method according to claim 14 wherein no portion of the device is in contact with an esophagus of the patient and further wherein no portion of the device is in contact with an abdominal wall of the patient.
